(19) United States
(12) Patent Application Publication
(10) Pub. No.: US 2009/0036325 A1
(43) Pub. Date: Feb. 5, 2009

(54) Directed assembly of amplicons to enhance read pairing signature with massively parallel short read sequencers

(75) Inventors: Kevin J. McKernan, Marblehead, MA (US); John Andrew Sheridan, Marblehead, MA (US); Douglas R. Smith, Gloucester, MA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 12/154,294

(22) Filed: May 21, 2008

Related U.S. Application Data
(60) Provisional application No. 60/931,752, filed on May 25, 2007.

(51) Int. Cl. C40B 50/06; C12P 19/34

(52) U.S. Cl. 506/26; 435/91.2

(57) ABSTRACT

The present teachings relate to improved methods, kits, and compositions for making nucleic acid libraries and sequencing nucleic acids. In some embodiments, directionally defined concatamers are generated, facilitating sequencing efforts.

### FIELD

The present teachings generally relate to methods of nucleic acid library construction and nucleic acid sequencing.

### INTRODUCTION

PCR amplification of genes in the human genome for resequencing is often performed as 500 bp amplification of the exons of interest. Since the exons of a gene are often distributed over 100 kb or more, amplifying the entire gene in one amplicon is currently unobtainable with current PCR technology. Instead the gene is amplified as many small (500 bp) amplicons covering each independent exon or a few closely located exons into 1 amplicon.

Gene families are often the target for many resequencing projects (See NCI Human Cancer Genome Project). These gene families often have highly similar sequences since they are homologs and functionally similar. For example, there are 90 Tyrosine Kinases in the Human Genome targeted by the Human Cancer Genome Project. Recently, there has been the emergence of commercially available massively parallel short read DNA sequencers such as Solexa, Helicos, 454 and Applied Biosystems. These sequencers do not segregate sequencing samples in predefined adressable wells but instead pool collections of molecules into a common flow cell for sequencing. As a result thousands to millions of amplicons are sequenced in parallel and it can be difficult to segregate reads derived from 2 highly similar genes or amplicons, since there is no apriori knowledge of which amplicon exists at a given feature (as there is with capillary electrophoresis sequencing).

Traditional whole genome shotgun sequencing relied on paired end reads (or mate pairs) to resolve such ambiguities since the paired read would often be derived from DNA thousands of bases away and could contribute more signature to any paired set of reads which was derived from highly homologous sequence. Creating paired end reads derived from, and solely constrained to, 500 bp amplicons provides less signature since the reads are so closely located in the genome.

Additionally, many of these novel massively parallel DNA sequencers only produce short reads (20-100 bp, see for example Margulies et al, Nature, Vol 437, Sep. 15, 2005) thus providing less signature than a paired read would provide. Shorter reads also require the production of far more amplicons (20 bp reads would suggest one needs an amplicon every 20 bp in the gene to get full sequence coverage) which demands more PCR primers and shorter amplicons.

To simplify the primer design one can still generate 500 bp amplicons and then shear the amplicons to facilitate ligation of universal adapters to the sheared fragments such that universal primers can be randomly attached. This could be performed on 1000s to millions of amplicons at a time and would provide random sequencing initiation start points throughout the molecule.

However, shearing 500 bp fragments into 100 bp fragments is not trivial and tends to under-represent the sequences on the ends of the 500 bp amplicon. A solution to this problem is to ligate all of the desired amplicons into one large contig and then shear the contig and adapt the molecules with universal primer sites. This was demonstrated by Yu et al (Genome Res., April 1997; 7: 353-358; doi:10.1101/gr.7.4.353) to simplify the shotgun sequencing process of multiple cDNAs. Since the cDNAs were fairly long fragments (several kb), no other modification was required to make more meaningful read pairings. As a result the cDNAs were randomly ligated together and the likelihood that each concatenated molecule had the same terminal bases was miniscule, thus eliminating the end bias.

### DESCRIPTION OF EXEMPLARY EMBODIMENTS

In this application, the use of the singular includes the plural unless specifically stated otherwise. In this application, the word “a” or “an” means “at least one” unless specifically stated otherwise. In this application, the use of “or” means “and/or” unless stated otherwise. Furthermore, the use of the term “including,” as well as other forms, such as “includes” and “included,” is not limiting.

The section headings used herein are for organizational purposes only and are not to be construed as limiting the subject matter described. All documents, or portions of documents, cited in this application, including but not limited to patents, patent applications, articles, books, and treatises are hereby expressly incorporated by reference in their entirety for any purpose. In the event that one or more of the incorporated documents defines a term that contradicts that term's definition in this application, this application controls.

### SOME DEFINITIONS

As used herein, the term “an informative desired subset” refers to a subset of a complex nucleic acid mixture, where the nucleic acid fragments comprising the informative desired subset contain a first sticky fragment and a second sticky fragment, wherein the orientation of the first sticky fragment and the second sticky fragment is determined. Thus, the “informative desired subset” contains information in addition to its sequence. This additional information is the determined orientation of the first sticky fragment to the second sticky fragment.

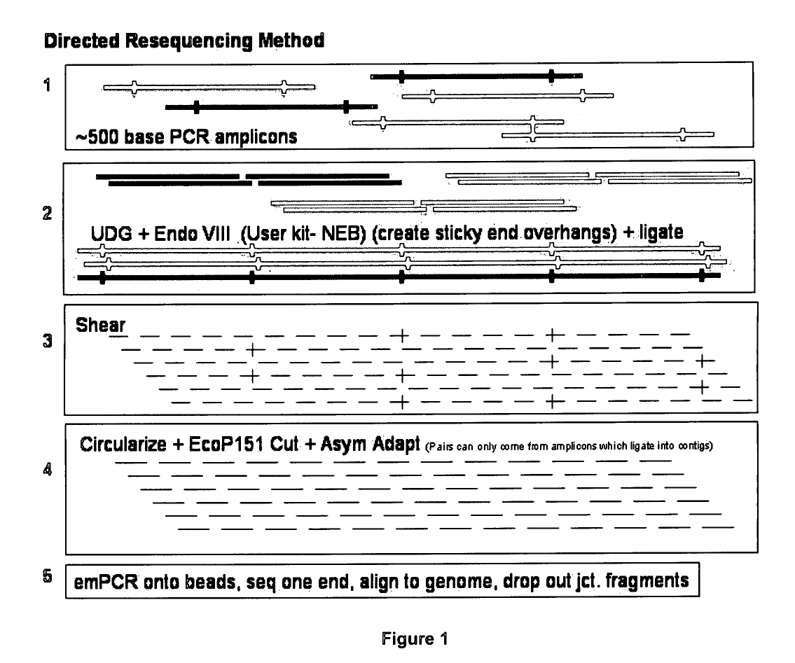

As used herein, the term “determined orientation” refers to the position of the 5′ end of a first sticky fragment to the 3′ end of a second sticky fragment, such that when the first sticky fragment and the second sticky fragment become ligated, a single 5′ to 3′ ordering is obtained. For example, the concatamer that results from the practicing of the present teachings contains a first sticky fragment with a downstream end ligated to the upstream end of a second sticky fragment. Further, such a concatamer does not contain the upstream end of the first sticky fragment ligated to the downstream end of the second sticky fragment. These scenarios are depicted in FIG. 1.

As used herein, the term “ligase” can comprise any number of enzymatic or non-enzymatic reagents. For example, ligase is an enzymatic ligation reagent that, under appropriate conditions, forms phosphodiester bonds between the 3′-OH and the 5′-phosphate of adjacent nucleotides in DNA molecules, RNA molecules, or hybrids. Temperature sensitive ligases, include, but are not limited to, bacteriophage T4 ligase and E. coli ligase. Thermostable ligases include, but are not limited to, Afu ligase, Taq ligase, Tfl ligase, Tth ligase, Tth HB8 ligase, Thermus species AK16D ligase and Pfu ligase (see for example Published P.C.T. Application WO00/26381, Wu et al., Gene, 76(2):245-254, (1989), Luo et al., Nucleic Acids Research, 24(15): 3071-3078 (1996). The skilled artisan will appreciate that any number of thermostable ligases, including DNA ligases and RNA ligases, can be obtained from thermophilic or hyperthermophilic organisms, for example, certain species of eubacteria and archaea; and that such ligases can be employed in the disclosed methods and kits. Further, reversibly inactivated enzymes (see for example U.S. Pat. No. 5,773,258) can be employed in some embodiments of the present teachings.

Chemical ligation agents include, without limitation, activating, condensing, and reducing agents, such as carbodiimide, cyanogen bromide (BrCN), N-cyanoimidazole, imidazole, 1-methylimidazole/carbodiimide/cystamine, dithiothreitol (DTT) and ultraviolet light. Autoligation, i.e., spontaneous ligation in the absence of a ligating agent, is also within the scope of the teachings herein. Detailed protocols for chemical ligation methods and descriptions of appropriate reactive groups can be found in, among other places, Xu et al., Nucleic Acid Res., 27:875-81 (1999); Gryaznov and Letsinger, Nucleic Acid Res. 21:1403-08 (1993); Gryaznov et al., Nucleic Acid Res. 22:2366-69 (1994); Kanaya and Yanagawa, Biochemistry 25:7423-30 (1986); Luebke and Dervan, Nucleic Acids Res. 20:3005-09 (1992); Sievers and von Kiedrowski, Nature 369:221-24 (1994); Liu and Taylor, Nucleic Acids Res. 26:3300-04 (1999); Wang and Kool, Nucleic Acids Res. 22:2326-33 (1994); Purmal et al., Nucleic Acids Res. 20:3713-19 (1992); Ashley and Kushlan, Biochemistry 30:2927-33 (1991); Chu and Orgel, Nucleic Acids Res. 16:3671-91 (1988); Sokolova et al., FEBS Letters 232:153-55 (1988); Naylor and Gilham, Biochemistry 5:2722-28 (1966); and U.S. Pat. No. 5,476,930.

Photoligation using light of an appropriate wavelength as a ligation agent is also within the scope of the teachings. In some embodiments, photoligation comprises probes comprising nucleotide analogs, including but not limited to, 4-thiothymidine (s⁴T), 5-vinyluracil and its derivatives, or combinations thereof. In some embodiments, the ligation agent comprises: (a) light in the UV-A range (about 320 nm to about 400 nm), the UV-B range (about 290 nm to about 320 nm), or combinations thereof, (b) light with a wavelength between about 300 nm and about 375 nm, (c) light with a wavelength of about 360 nm to about 370 nm; (d) light with a wavelength of about 364 nm to about 368 nm, or (e) light with a wavelength of about 366 nm. In some embodiments, photoligation is reversible. Descriptions of photoligation can be found in, among other places, Fujimoto et al., Nucl. Acid Symp. Ser. 42:39-40 (1999); Fujimoto et al., Nucl. Acid Res. Suppl. 1:185-86 (2001); Fujimoto et al., Nucl. Acid Suppl., 2:155-56 (2002); Liu and Taylor, Nucl. Acid Res. 26:3300-04 (1998) and on the world wide web at: sbchem.kyoto-u.ac.jp/saito-lab.

As used herein, the term “amplifying” refers to any means by which at least a part of a target polynucleotide, target polynucleotide surrogate, or combinations thereof, is reproduced, typically in a template-dependent manner, including without limitation, a broad range of techniques for amplifying nucleic acid sequences, either linearly or exponentially. Exemplary means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA) and the like, including multiplex versions or combinations thereof, for example but not limited to, OLA/PCR, PCR/OLA, LDR/PCR, PCR/PCR/LDR, PCR/LDR, LCR/PCR, PCR/LCR (also known as combined chain reaction-CCR), and the like. Descriptions of such techniques can, be found in, among other places, Sambrook et al. Molecular Cloning, 3^{rd} Edition; Ausbel et al.; PCR Primer: A Laboratory Manual, Diffenbach, Ed., Cold Spring Harbor Press (1995); The Electronic Protocol Book, Chang Bioscience (2002), Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. No. 6,027,998; U.S. Pat. No. 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol. Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. No. 5,830,711, U.S. Pat. No. 6,027,889, U.S. Pat. No. 5,686,243, Published P.C.T. Application WO0056927A3, and Published P.C.T. Application WO9803673A1. In some embodiments, newly-formed nucleic acid duplexes are not initially denatured, but are used in their double-stranded form in one or more subsequent steps. An extension reaction is an amplifying technique that comprises elongating a linker probe that is annealed to a template in the 5′ to 3′ direction using an amplifying means such as a polymerase and/or reverse transcriptase. According to some embodiments, with appropriate buffers, salts, pH, temperature, and nucleotide triphosphates, including analogs thereof, i.e., under appropriate conditions, a polymerase incorporates nucleotides complementary to the template strand starting at the 3′-end of an annealed linker probe, to generate a complementary strand. In some embodiments, the polymerase used for extension lacks or substantially lacks 5′ exonuclease activity. In some embodiments of the present teachings, unconventional nucleotide bases can be introduced into the amplification reaction products and the products treated by enzymatic (e.g., glycosylases) and/or physical-chemical means in order to render the product incapable of acting as a template for subsequent amplifications. In some embodiments, uracil can be included as a nucleobase in the reaction mixture, thereby allowing for subsequent reactions to decontaminate carryover of previous uracil-containing products by the use of uracil-N-glycosylase (see for example Published P.C.T. Application WO9201814A2). In some embodiments of the present teachings, any of a variety of techniques can be employed prior to amplification in order to facilitate amplification success, as described for example in Radstrom et al., Mol. Biotechnol. 2004 February; 26(2):133-46. In some embodiments, amplification can be achieved in a self-contained integrated approach comprising sample preparation and detection, as described for example in U.S. Pat. Nos. 6,153,425 and 6,649,378. Reversibly modified enzymes, for example but not limited to those described in U.S. Pat. No. 5,773,258, are also within the scope of the disclosed teachings. The present teachings also contemplate various uracil-based decontamination strategies, wherein for example uracil can be incorporated into an amplification reaction, and subsequent carry-over products removed with various glycosylase treatments (see for example U.S. Pat. No. 5,536,649, and U.S. Provisional Application 60/584,682 to Andersen et al.). Those in the art will understand that any protein with the desired enzymatic activity can be used in the disclosed methods and kits. Descriptions of DNA polymerases, including reverse transcriptases, uracil N-glycosylase, and the like, can be found in, among other places, Twyman, Advanced Molecular Biology, BIOS Scientific Publishers, 1999; Enzyme Resource Guide, rev. 092298, Promega, 1998; Sambrook and Russell; Sambrook et al.; Lehninger; PCR: The Basics; and Ausbel et al.

The present teachings provide a more informative approach for nucleic acid sequencing. The approach is to ligate the molecules in a directed predetermined manner such that an expected order and orientation of the molecules is created. This makes the mate pairing synthetic but still meaningful, in the sense that the experimentalist knows the ordering of the sequence information in the genome. In some embodiments, it is preferable for the directed ligation to be compatible with single tube massively parallel ligation such that thousands to millions of amplicons could be ligated in parallel and in a single tube. Thus, the present teachings provide a method for creating sticky end PCR products, such that the sticky ends are predetermined barcode sequences which direct the ligation of amplicons together, and thus facilitate the assembly of amplicons into molecules containing fragments in a predetermined order.

In light of the present teachings, and in view of the approaches for gene assembly and cloning described in Donahue (Nucleic Acids Research, 2002, Vol. 30, No. 18 e95) that are readily available in the art, one of skill in the art can now organize molecules into a predefined arrangement which provides the most signature for short read sequences once sheared and adapted for random shotgun assembly. No efforts to conserve coding frame or build genes is intended and in fact it may be preferred to include introns in the sequence.

There are two alternative assembly strategies to Donahue et al, both of which can be applied in the context of the present teachings. The first makes the use of a uracil group in the PCR primer site. This specific base can be cleaved with UDG and Endonuclease VIII to generate a phosphate sticky end overhang. This can also be performed with Inosine and the enzyme EndoV. NEB provides a commercially available kit which performs this UDG and EndoVIII as a cloning kit. Both of these approaches suffer in that the cleavage must occur at either a Uracil or Inosine base and require long enzymatic steps to perform. Thus, a preferred solution is a chemically cleavable base which is independent of the base sequence, thus facilitating more freedom in oligo design (not restricted to Uracil sequences for the cut site). The use of a phosphorothiolate linkage in the primer can serve as a cleavage loci and it is independent of the base analog. This bond is cleaved by aqueous Silver in minutes. A variety of sequencing approaches can be used in the context of the present teachings, as found for example in Published US Patent Applications US20070026438A1 and US20060024681A1. Additionally, any of a variety of methods can be employed for isolating the nucleic acids in the context of the present teachings, as found for example in Published US Patent Applications US20070054285A1, US20060177836A1, US20060078923A1, US20060024701A1, and US20040197780A1, as well as U.S. Pat. No. 6,534,262.

Thus, the present teachings provide a way of generating concatameric molecules containing a collection of fragments in the same orientation. These concatamers can then be sheared, thus forming smaller fragments suitable for sequencing. By shearing after the concatenation, the problem of middle-bias that occurs when shearing smaller fragments is overcome. Placing the sheared fragments into a mate-pair configuration gives the experimentalist a piece of information, directionality, which can be used to facilitate informatic assembly of the genome.

FIG. 1 depicts one illustrative embodiment according to the present teachings.

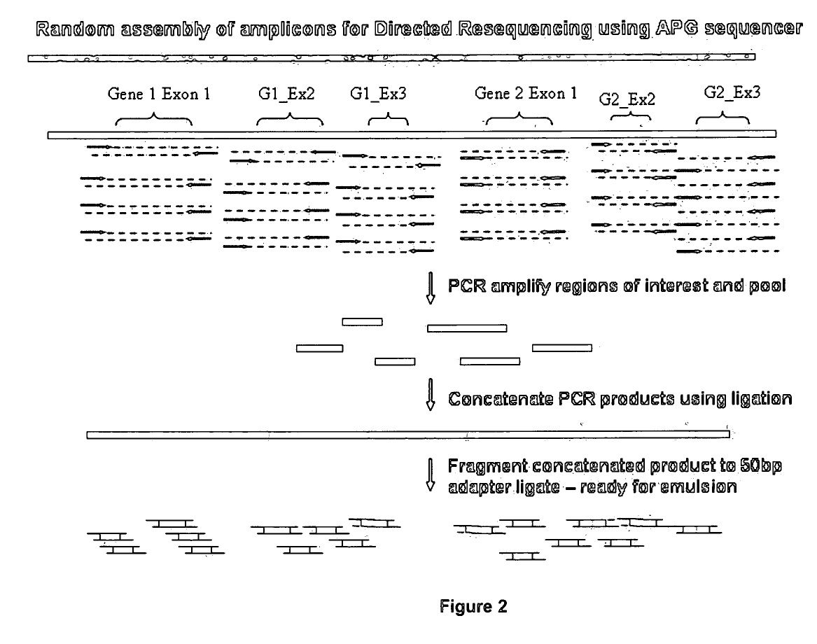

FIG. 2 depicts one illustrative embodiment according to the present teachings.

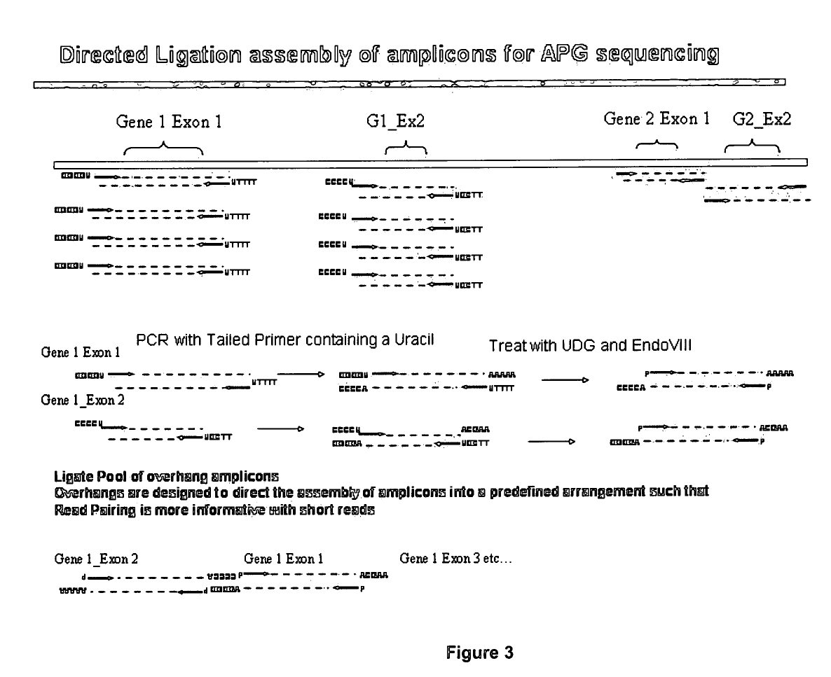

FIG. 3 depicts one illustrative embodiment according to the present teachings.

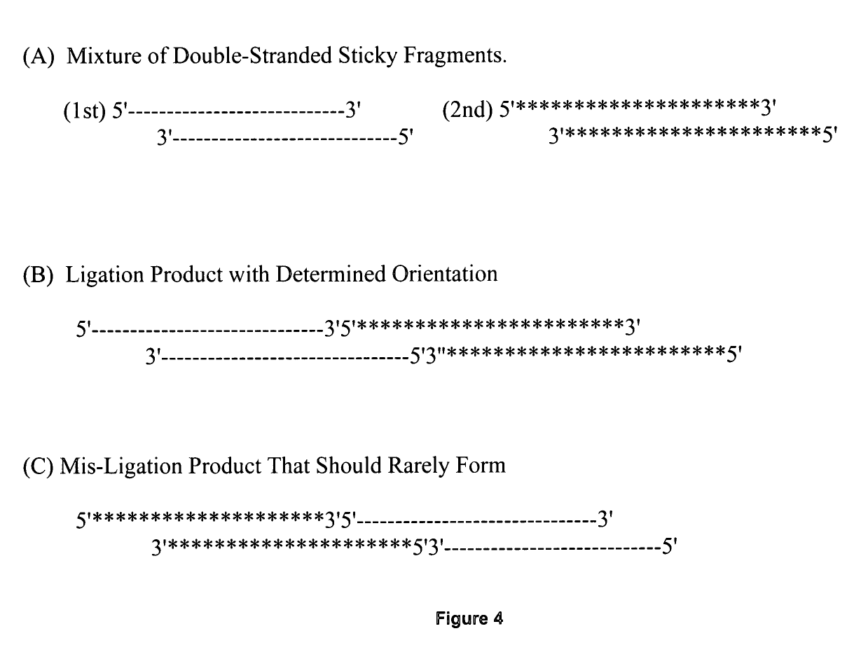

FIG. 4 depicts one illustrative embodiment according to the present teachings.

Approaches for making nucleic acid libraries are generally known in the art, and can be found described, for example, in U.S. Pat. No. 5,663,062, U.S. Pat. No. 5,858,731, and U.S. Pat. No. 6,641,998.

Exemplary Kits in Accordance with Some Embodiments of the Present Teachings

In some embodiments, the present teachings also provide kits designed to expedite performing certain methods. In some embodiments, kits serve to expedite the performance of the methods of interest by assembling two or more components used in carrying out the methods. In some embodiments, kits may contain components in pre-measured unit amounts to minimize the need for measurements by end-users. In some embodiments, kits may include instructions for performing one or more methods of the present teachings. In certain embodiments, the kit components are optimized to operate in conjunction with one another.

While the present teachings have been described in terms of these exemplary embodiments and experimental data, the skilled artisan will readily understand that numerous variations and modifications of these exemplary embodiments are possible without undue experimentation. All such variations and modifications are within the scope of the current teachings.

The present teachings are further discussed in the following claims.

1. A method of obtaining an informative desired subset of a complex nucleic acid mixture comprising;
amplifying the desired subset with a plurality of primer pairs to form a plurality of first amplicons, wherein each of the plurality of primer pairs comprises a forward primer and a reverse primer,
(a) wherein the forward primer comprises a first asymmetric adapter, and,
(b) wherein the reverse primer comprises a second asymmetric adapter;
cleaving the first asymmetric adapter and the second asymmetric adapter of the plurality of first amplicons to form a plurality of sticky fragments comprising a first sticky end and a second sticky end;
ligating the plurality of sticky fragments to form a concatamer comprising a plurality of sticky fragments bearing a determined orientation;
cleaving the concatamer to form a plurality of cleavage products; and,
obtaining an informative desired subset of the complex nucleic acid mixture.

2. The method of claim 1
(a) wherein the concatamer comprises a first sticky fragment with a downstream end ligated to an upstream end of a second sticky fragment, and,
(b) wherein the concatamer does not comprise the upstream end of the first sticky fragment ligated to the downstream end of the second sticky fragment.

3. The method of claim 2 further comprising;
amplifying the informative desired subset in an emulsion PCR, wherein the emulsion PCR comprises primer-immobilized beads, to form a collection of amplicon-bearing beads;
immobilizing the amplicon-bearing beads on a solid support; and,
sequencing the amplicon of each amplicon-bearing bead.

4. The method according to claim 3 wherein the sequencing comprising ligation-sequencing.

5. The method according to claim 3 wherein the sequencing comprises sequencing by synthesis.

6. A method of forming a concatamer containing sticky fragments comprising;
amplifying a complex nucleic acid sample with a plurality of primer pairs to form a plurality of first amplicons, wherein each of the plurality of primer pairs comprises a forward primer and a reverse primer,
(a) wherein the forward primer comprises a first asymmetric adapter, and,
(b) wherein the reverse primer comprises a second asymmetric adapter;
cleaving the first asymmetric adapter and the second asymmetric adapter of the plurality of first amplicons to form a plurality of sticky fragments comprising a first sticky end and a second sticky end; and,
ligating the plurality of sticky fragments to form a concatamer comprising a plurality of sticky fragments bearing a determined orientation,
wherein the ligating does not comprise a vector, and
wherein the first asymmetric adapter, the second asymmetric adapter, or both the first and the second asymmetric adapter comprise a phosphorothiolate and wherein the cleaving comprises treating with aqueous Silver.
